(19) United States
(12) Patent Application Publication
(10) Pub. No.: US 2005/0136465 A1
(43) Pub. Date: Jun. 23, 2005

(54) Screening assays

(76) Inventors: Roger G. Clerc, Binningen (CH); Guillemette Duchateau-Nguyen, Riedisheim (FR); Christophe Gardes, Bartenheim (FR); Jacques Mizrahi, Bottmingen (CH); Claes-Goran Ostenson, Stockholm (SE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/019,829

(22) Filed: Dec. 22, 2004

(30) Foreign Application Priority Data
Dec. 22, 2003 (EP) 03104902.6

(51) Int. Cl. C12Q 1/68

(52) U.S. Cl. 435/6

(57) ABSTRACT

The present invention relates to novel targets for identifying compounds that may be useful for the prevention and treatment of obesity.

Multifactorial diseases such as obesity are caused by mutations in more than one gene with a large contribution from environmental factors. There has been spectacular success in identifying the genes responsible for Mendelian disorders, whereas finding the susceptibility genes involved in multifactorial diseases has so far been difficult. The evidence suggests that humans inherit a genetic predisposition to gain weight on a high fat diet. Therefore optimizing patient sampling for the collection of tissues on the bases of clinical and physiological parameters is critical.

There is clearly an unmet medical need for novel therapeutic solutions to this health problem, in particular in the light of the fact that current medication that promote weight loss are transient as the lost excess of weight is gained back within 1 to 5 years. Therefore, there is a need to identify new targets for the development of new treatments.

### SUMMARY OF THE INVENTION

The invention provides methods (also referred to herein as “screening assays”) for identifying compounds which can be used for the modulation of body weight, e.g., for the treatment of a body weight disorder by detecting the effect of a test compound on the expression of the genes set forth in Seq ID Nos. 1-12 and 25-85 and/or the effect on the level of activity of the polypeptides set forth in Seq ID Nos. 13-24 and 86-146.

### DETAILED DESCRIPTION OF THE INVENTION

A set of 8000 patients, enrolled in a Diabetes/Obesity Prevention Program with the Stockholm Prevention Program, was monitored for a number of clinical parameters and vital signs. From this large pool of patients, a clinically well annotated series of tissue biopsies from 10 obese, non diabetic, and 10 matched control patients were analyzed for gene expression profiling. The following matched clinical parameters and vital signs were, among others, used to recruit these patients: BMI (control mean=22.2 sd±1.3 and case mean=32.8 sd±2.1), age (control mean=54.6 years and case mean=56.3 years), male gender, VO2 ratio, total fat versus truncal fat, waist-hip ratio, energy expediture, blood pressure, FA oxidation. CHO oxydation, OGTT negative, birth weight, no diabetes in the family, no smoking, sedentary and no alcohol habits.

TABLE 1

| Obese | Control |
|---|---|
| BMI 30-35 | BMI 20-23 |
| without impaired OGTT | without impaired OGTT |
| without type 2 diabetes | without type 2 diabetes |

Other parameters used for patient selection:
family history of diabetes
birth weight
blood pressure
medication (if any)
food intake
physical activity education
bodyweight history
chronic illness
tobacco and alcohol use
housing conditions
socio-economical factors

The methods provided by this invention entail identifying candidate or test compounds or agents (e.g., peptides, peptidomimetics, small molecules or other drugs) which bind a polypeptide selected from the group consisting of the polypeptides of Seq ID No. 13 to 24, or a polypeptide selected from the group consisting of the polypeptides of Seq ID No.86 to 146 and/or have a stimulatory or inhibitory effect on the activity or the expression of a polypeptide selected from the group consisting of the polypeptides of Seq ID No. 13 to 24, or a polypeptide selected from the group consisting of the polypeptides of Seq ID No. 86 to 146 and then determining which of the compounds that bind a polypeptide selected from the group consisting of the polypeptides of Seq ID No. 13 to 24 or a polypeptide selected from the group consisting of the polypeptides of Seq ID No.86 to 146 or have a stimulatory or inhibitory effect on the activity or the expression of a polypeptide selected from the group consisting of the polypeptides of Seq ID No. 13 to 24 or a polypeptide selected from the group consisting of the polypeptides of Seq ID No.86 to 146 have an effect on the feeding behavior, body weight, or metabolic rate of a mammal (e.g., a mouse or a rat) in an in vivo assay.

The present invention pertains to a method of screening for compounds that reduce and/or prevent obesity comprising: a) contacting a cell expressing a gene listed in table 2 with a compound; and b) measuring the expression of said gene, or a polypeptide encoded by said gene; wherein a compound which up-regulates expression is a compound which causes an increase of expression of said gene or of the polypeptide encoded by said gene.

The term “up-regulation of expression” as used herein refers to an increase in expression of mRNA levels of a nucleic acid, or to an increase in expression of polypeptide levels. This term may also relate to increased post-translational modifications that are necessary for the activity and/or function of a polypeptide, e.g. addition of sugar moieties, phosphorylation etc.

A cell used in the method hereinbefore described, or in any of the methods hereinafter described may be an adipocyte, or a host or host cell as defined hereinafter.

Preferably, said gene is Seq ID. No.1. In another preferred embodiment, said gene is Seq ID No. 2. In another preferred embodiment, said gene is Seq ID No. 3. In another preferred embodiment, said gene is Seq ID No. 4. In another preferred embodiment, said gene is Seq ID No. 5. In another preferred embodiment, said gene is Seq ID No. 6.

The present invention further provides a method of screening for compounds that reduce and/or prevent obesity comprising: a) contacting a cell expressing a gene selected from the group consisting of Seq ID No. 25-37 with a compound; and b) measuring the expression of said gene, or a polypeptide encoded by said gene;
wherein a compound which up-regulates gene expression is a compound which causes an increase of expression of said gene or of the polypeptide encoded by said gene.

Preferably, said polypeptide is Seq ID No. 13. In another preferred embodiment, said polypeptide is Seq ID No. 14. In another preferred embodiment, said polypeptide is Seq ID No. 15. In another preferred embodiment, said polypeptide is Seq ID No. 16. In another preferred embodiment, said polypeptide is Seq ID No. 17. In another preferred embodiment, said polypeptide is Seq ID No. 18.

The present invention also pertains to a method of screening for compounds that reduce and/or prevent obesity comprising a) contacting a cell expressing a gene listed in table 3 with a compound; and b) measuring the expression of said gene, or a polypeptide encoded by said gene;
wherein a compound which down-regulates gene expression is a compound which causes a decrease of said gene or a polypeptide encoded by said gene.

The term “down-regulation of expression” as used herein refers to a decrease in expression of mRNA levels of a nucleic acid, or to a decrease in expression of polypeptide levels. This term may also relate to decreased post-translational modifications that are necessary for the activity and/or function of a polypeptide, e.g. addition of sugar moieties, phosphorylation etc.

Preferably, said gene is Seq ID. No.7. In another preferred embodiment, said gene is Seq ID No. 8. In another preferred embodiment, said gene is Seq ID No. 9. In another preferred embodiment, said gene is Seq ID No. 10. In another preferred embodiment, said gene is Seq ID No. 11. In another preferred embodiment, said gene is Seq ID No. 12.

Preferably, said polypeptide is Seq ID. No.19. In another preferred embodiment, said polypeptide is Seq ID No. 20. In another preferred embodiment, said polypeptide is Seq ID No. 21. In another preferred embodiment, said polypeptide is Seq ID No. 22. In another preferred embodiment, said polypeptide is Seq ID No. 23. In another preferred embodiment, said polypeptide is Seq ID No. 24.

The present invention further provides a method of screening for compounds that reduce and/or prevent obesity comprising: a) contacting a cell expressing a gene selected from the group consisting of Seq ID No. 38 to 85 with a compound; and b) measuring the expression of said gene, or a polypeptide encoded by said gene;
wherein a compound which down-regulates gene expression is a compound which causes a decrease of said gene or a polypeptide encoded by said gene.

The present invention provides a method of screening for compounds that reduce and/or prevent obesity comprising: a) contacting a polypeptide selected from the group consisting of Seq ID No. 13 to 18 with a compound; and b) determining and/or measuring the activity of said polypeptide; wherein a compound which reduces and/or prevents obesity by agonizing said polypeptide is a compound which causes an increase in activity of said polypeptide.

The present invention also pertains to a method for screening of compounds that reduce and/or prevent obesity comprising: a) contacting a cell expressing a nucleic acid encoding a polypeptide selected from the group consisting of Seq ID No. 13 to 18 with a compound; and b) determining the activity of said polypeptide; wherein a compound which reduces and/or prevents obesity by agonizing said polypeptide is a compound which causes an increase in activity of said polypeptide.

Preferably, said polypeptide is Seq ID. No.13. In another preferred embodiment, said polypeptide is Seq ID No. 14. In another preferred embodiment, said polypeptide is Seq ID No. 15. In another preferred embodiment, said polypeptide is Seq ID No. 16. In another preferred embodiment, said polypeptide is Seq ID No. 17. In another preferred embodiment, said polypeptide is Seq ID No. 18.

The terms “activity ” as used with respect to the polypeptide encoded by Seq ID No. 1, AZGP1, refers to the lipolytic and lipid mobilizing activity of AZGP1. Assays to determine these activities are well known in the art and are e.g. described in W099/62939.

The terms “activity ” as used with respect to the polypeptide encoded by Seq ID No. 2, IRS 1, refers to modifications of IRS1, eg. by phosphorylation, preferably by tyrosine phosphorylation, and/or binding to downstream effectors following activation, eg. binding to PI3-kinase, Syp or Grb2, and/or phosphorylation or activation of downstream effectors of IRS1 activation. Assays to measure the activity of IRSI are well known in the art, and are, for example, described in Ridderstrale et al., J. Biol. Chem. 270, 3471, 1995; or Kuhne et al., J. Biol. Chem. 268, 11479-81, 1993.

The terms “activity ” as used with respect to the polypeptide encoded by Seq ID No. 3, ApoAI, refers to the binding of ApoAl and/or lipid vesicles containing ApoAl to target cells and/or the ability of ApoAl and/or lipid vesicles containing ApoAl to induce efflux of cellular cholesterol and phospholipids. Assays to determine these activities are well known in the art and are e.g. described in Hauser et al., Biochemistry 1998, 37, 17843-17850; Yancey et al., Biochemistry 1995, 34, 7955-7965.

The terms “activity ” as used with respect to the polypeptide encoded by Seq ID No. 4, ARNT, refers to the transcriptional and/or co-activating activity of ARNT. Assays to determine these activities are well known in the art and are e.g. described in Whitelaw et al., Mol. Cell Biol. 12, 1994, 8343-8355 and/or Brunnberg et al., Proc. Natl. Acad. Sci. USA 100, 2003, 6517-6522.

The terms “activity ” as used with respect to the polypeptide encoded by Seq ID No. 5, creatine kinase from brain (CKB), refers to the kinase activity of CKB. Assays to determine these activities are well known in the art and are e.g. described in O'Gorman et al., Biochem. Biophys. Acta 1276, 1996, 161-170; and/or Durany et al., Mol. Pathol. 55, 2002, 242-249.

The terms “activity ” as used with respect to the polypeptide encoded by Seq ID No. 6, 6 sterol-c5-desaturase, refers to the ability of 6 sterol-c5-desaturase to desaturate n-3 and n-6 fatty acids. Assays to determine these activities are well known in the art and are e.g. described in de Antueno et al., FEBS Letters 509, 2001, 77-80; de Antueno et al., FEBS Letters 491, 2001, 247-251; Taton et al., Biochemistry 39, 2000, 701-711.

The present invention also pertains to a method of screening for compounds that reduce and/or prevent obesity comprising: a) contacting a polypeptide selected from the group consisting of Seq ID No. 86 to 98 with a compound; and b) determining the activity of said polypeptide;
wherein a compound which reduces and/or prevents obesity by agonizing said polypeptide is a compound which causes an increase in activity of said polypeptide.

A method for screening of compounds that reduce and/or prevent obesity comprising: a) contacting a cell expressing a nucleic acid encoding a polypeptide selected from the group consisting of Seq ID No. 86 to 98 with a compound; and b) determining the activity of said polypeptide;
wherein a compound which reduces and/or prevents obesity by agonizing said polypeptide is a compound which causes an increase in activity of said polypeptide.

The present invention pertains to a method for screening of compounds that reduce and/or prevent obesity comprising: a) contacting a cell expressing a polypeptide selected from the group consisting of Seq ID No. 19 to 24 with a compound; and b) determining the activity of said gene, or a polypeptide encoded by said gene; wherein a compound which reduces and/or prevents obesity by antagonizing said polypeptide is a compound which causes a decrease in activity of said polypeptide.

The present invention provides a method for screening of compounds that reduce and/or prevent obesity comprising: a) contacting a cell expressing a nucleic acid encoding a polypeptide selected from the group consisting of Seq ID No. 19 to 24 with a compound; and b) determining the activity of said polypeptide; wherein a compound which reduces and/or prevents obesity by antagonizing said polypeptide is a compound which causes a decrease in activity of said polypeptide.

Preferably, said polypeptide is Seq ID. No.19. In another preferred embodiment, said polypeptide is Seq ID No. 20. In another preferred embodiment, said polypeptide is Seq ID No. 21. In another preferred embodiment, said polypeptide is Seq ID No. 22. In another preferred embodiment, said polypeptide is Seq ID No. 23. In another preferred embodiment, said polypeptide is Seq ID No. 24.

The terms “activity ” as used with respect to the polypeptide encoded by Seq ID No. 7, osteomodulin (osteoadherin), refers to the ability of osteomodulin to mediate cell adhesion and/or attachment. Assays to determine these activities are well known in the art and are e.g. described in Wendel et al., J. Cell Biol. 141, 1998, 839-847.

The terms “activity ” as used with respect to the polypeptide encoded by Seq ID No. 8, VLDLR (Very Low Density Lipoprotein Receptor), refers to the ability of VLDLR to e.g. bind bind to ligands such as proteinases, apolipoproteins, extracellular proteins. Assays to determine these activities are well known in the art and are e.g. described in Rettenberger et al., J. Biol. Chem. 274, 1999, 8973-8980, which also lists references for binding assays for ligands including serine proteinase-serpin complexes, the pro-enzyme form of uPA, ApoE containing lipoproteins, apo(a), lipoprotein lipase and thrombospondin-1.

The terms “activity ” as used with respect to the polypeptide encoded by Seq ID No. 9,11-beta-hydroxysteroid dehydrogenase type 1 (HSD11B1), refers to dehydrogenase and/or reductase activities of HSD11B1. Assays to determine these activities are well known in the art and are e.g. described in Bujalska, I. J. et al., J. Clin. Endocrinol. Metab. 2002, 87, 1205-1210.

The terms “activity ” as used with respect to the polypeptide encoded by Seq ID No. 10, reelin, refers to the ability of reelin to bind to receptors, e.g. VLDLR or ApoER2, or to induce phosphorylation of downstream signaling molecules, e.g. Disabled 1. Assays to determine these activities are well known in the art and are e.g. described in D'Arcangelo, G. et al., Neuron 1999, 24, 471-479, and Howell, B. W. et al., Genes Dev. 1999, 13, 643-648.

The terms “activity ” as used with respect to the polypeptide encoded by Seq ID No. 11, Multidrug resistance protein 7 (MRP7, ABCC10), refers to the transporter activities of ABCC10. Assays to determine these activities are well known in the art and are e.g. described in Mol. Pharmacol. 2003, 63, 351-358.

The terms “activity ” as used with respect to the polypeptide encoded by Seq ID No. 12, cyclophilin 40 (CyP40), refers to the ability of CyP40 to bind to other chaperones, e.g. Hsp90, and to its chaperone activity. Assays to determine these activities are well known in the art and are e.g. described in Ward, B.K., et al., J. Biol. Chem. 2002, 277, 40799-40809, and Freeman B. C. et al., Science 1996, 274, 1718-1720.

The present invention provides a method of screening for compounds that reduce and/or prevent obesity comprising: a) contacting a polypeptide selected from the group consisting of Seq ID No. 99 to 146 with a compound; and b) determining the activity of said polypeptide;
wherein a compound which reduces and/or prevents obesity by antagonizing said polypeptide is a compound which causes a decrease in activity of said polypeptide.

The present invention further provides a method for screening of compounds that reduce and/or prevent obesity comprising: a) contacting a cell expressing a nucleic acid encoding a polypeptide selected from the group consisting of Seq ID No. 99 to 146 with a compound; and b) determining the activity of said polypeptide;
wherein a compound which reduces and/or prevents obesity by antagonizing said polypeptide is a compound which causes a decrease in activity of said polypeptide.

The present invention also provides a method of screening for compounds that bind to a polypeptide selected from the group consisting of the polypeptides of Seq ID No. 13 to 24, comprising the steps of a) contacting a compound with said polypeptide; and b) determining the ability of said compound to bind to said polypeptide.

The present invention provides a method of screening for compounds that bind to a polypeptide selected from the group consisting of the polypeptides of Seq ID No. 86 to 146, comprising the steps of a) contacting a compound with said polypeptide; and b) determining the ability of said compound to bind to said polypeptide.

Candidate or test compounds or agents which bind a polypeptide selected from the group consisting of Seq ID No 13 to 24 or a polypeptide selected from the group consisting of Seq ID No 86 to 146 and/or have a stimulatory or inhibitory effect on the activity or the expression of said polypeptide are identified in assays that employ either cells which express a form of said polypeptide (cell-based assays) or isolated polypeptide (cell-free assays). The various assays can employ a variety of forms of said polypeptide (e.g., full-length polypeptide, a biologically active fragment of a polypeptide, or a fusion protein which includes all or a portion of said polypeptide). Moreover, the polypeptide can be derived from any suitable mammalian species. The assay can be a binding assay entailing direct or indirect measurement of the binding of a test compound or the polypeptide to a known ligand or receptor, as defined above. The assay can also be an activity assay entailing direct or indirect measurement of the activity of said polypeptide. The assay can also be an expression assay entailing direct or indirect measurement of the expression of said polypeptide (e.g., polypeptide- encoding mRNA or the polypeptide). The various screening assays are combined with an in vivo assay entailing measuring the effect of the test compound on the feeding behavior, body weight, or metabolic rate of a mammal (e.g., a mouse or a rat).

In another embodiment, the assay is a cell-based assay comprising contacting a cell expressing a polypeptide (e.g., full-length polypeptide, a biologically active fragment of said polypeptide, or a fusion protein which includes all or a portion of said polypeptide) with a test compound and determining the ability of the test compound to modulate (e.g., stimulate or inhibit) the activity of the polypeptide. Determining the ability of the test compound to modulate the activity of the said polypeptide can be accomplished by any method suitable for measuring the activity of said polypeptide.

The present invention also includes cell-free assays. Such assays involve contacting a form of a polypeptide selected from the group consisting of Seq ID No 13 to 24 or a polypeptide selected from the group consisting of Seq ID No 86 to 146 (e.g., full-length polypeptide, a biologically active fragment of said polypeptide, or a fusion protein comprising all or a portion of said polypeptide) with a test compound and determining the ability of the test compound to bind to said polypeptide. Binding of the test compound to said polypeptide can be determined either directly or indirectly as described above. In one embodiment, the assay includes contacting the said polypeptide with a known compound which binds said polypeptide to form an assay mixture, contacting the assay mixture with a test compound, and determining the ability of the test compound to interact with said polypeptide, wherein determining the ability of the test compound to interact with said polypeptide comprises determining the ability of the test compound to preferentially bind to the said polypeptide as compared to the known compound.

The cell-free assays of the present invention are amenable to use of either a membrane-bound form of a polypeptide or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-dodecylmaltoside, octanoyl-N-mcthylglucamidc, decanoyl-Nmethylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N, N-dimethyl-3-ammonio-1-propane sulfonate.

In various embodiments of the above assay methods of the present invention, it may be desirable to immobilize a polypeptide to facilitate separation of complexed from uncomplexed forms of the polypeptide with a binding molecule, as well as to accommodate automation of the assay. Binding of a test compound to a polypeptide, or interaction of a polypeptide with a binding molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed binding protein or polypeptide, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of a polypeptide hereinbefore described can be determined using standard techniques.

j Other techniques for immobilizing proteins on matrices can also be used in the screening assays of the invention. For example, either a polypeptide hereinbefore described or its binding molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with a polypeptide or binding molecules, but which do not interfere with binding of the polypeptide of the invention to its binding molecule, can be derivatized to the wells of the plate. Unbound binding protein or polypeptide of the invention is trapped in the wells by antibody conjugation. Methods for detecting such complexes, in addition to those described above for the GST-immobilized complexes, include immunodetection of complexes using antibodies reactive with a polypeptide hereinbefore described or binding molecule, as well as enzyme-linked assays which rely on detecting an enzymatic activity associated with a polypeptide or binding molecule.

I. Test Compounds

Suitable test compounds for use in the screening assays of the invention can be obtained from any suitable source, e.g., conventional compound libraries. The test compounds can also be obtained using any of the numerous approaches in combinatorial library methods known in the art, including biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb Ct al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew Chem. Int. Ed. Engl. 33:2059; Carell et al. (1994) Angew Chem. Int. Ed. Engi.33:2061; and Gallop et al. (1994) J. Med. Chem. 37:1233.

Libraries of compounds may be presented in solution (e.g., Houghten (1992) BioTechniques 13.412-421), or on beads (Lam (1991) Nature 354:82-84), chips (Fodor (1993) Nature 364:555-556), bacteria (U.S. Pat. No.5,223,409), spores (U.S. Pat. Nos. 5,571,698; 5,403,484; and 5,223, 409), plasmids (Cull et al. (1992) Proc. Natl. Acad. Sci. USA 89.1865-1869) or phage (Scott and Smith (1990) Science249:386-390; Devlin (1990) Science 249:404-406; Cwirla et al. (1990) Proc. Natl. Acad. Sci. USA 87:6378-6382; and Felici (1991) J. Mol. Biol. 222:301-310).

The present invention provides a compound identified by any of the methods hereinbefore described.

II. Isolated Nucleic Acid Molecules

One aspect of the invention pertains to isolated nucleic acid molecules that encode a polypeptide selected from the group consisting of Seq ID No. 13 to 24 a polypeptide selected from the group consisting of Seq ID No 86 to 146 or a biologically active portion thereof, as well as nucleic acid molecules sufficient for use as hybridization probes to identify nucleic acid molecules encoding a gene selected from the group consisting of Seq ID No. 1 to 12 or a gene selected from the group consisting of Seq ID No 25 to 85 and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. This section describes the nucleic acids hereinbefore described and methods for making and using such nucleic acids.

An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention can be isolated using standard molecular biology techniques and the sequence information provided herein. Using all or a portion of the nucleic acid sequences selected from the group consisting of Seq ID No. 1 to 12 or a gene selected from the group consisting of Seq ID No 25 to 85 as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y, 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

Moreover, a nucleic acid molecule of the invention can comprise only a portion of a nucleic acid sequence encoding a polypeptide selected from the group consisting of Seq ID No. 13 to 24 or a polypeptide selected from the group consisting of Seq ID No 86 to 146, for example, a fragment which can be used as a probe or primer or a fragment encoding a biologically active portion of a polypeptide selected from the group consisting of Seq ID No.13 to 24 or a polypeptide selected from the group consisting of Seq ID No 86 to 146. The nucleotide sequence determined from the cloning of any one of the genes listed in tables 2 and 3 or Seq ID No. 25 to 85 for the generation of probes and primers designed for use in identifying and/or cloning allelic variants and other variants of any one of the genes listed in tables 2 and 3 or Seq ID No. 25 to 85. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or antisense sequence of any one of the genes listed in tables 2 and 3 or Seq ID No. 25 to 85 or naturally occurring mutant or allelic variant thereof.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which miss-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a “biologically active portion” of a polypeptide selected from the group consisting of Seq ID No. 13 to 24 or a polypeptide selected from the group consisting of Seq ID No 86 to 146 can be prepared by isolating a portion of nucleic acid which encodes a polypeptide having a biological activity, expressing the encoded portion of the polypeptide protein (e.g., by recombinant expression in vitro) and assessing the activity of the encoded portion of the polypeptide.

The invention further encompasses nucleic acid molecules that differ from the nucleotide sequence of a nucleic acid selected from the group consisting of Seq ID No. 1 to 12 or a gene selected from the group consisting of Seq ID No 25 to 85 due to degeneracy of the genetic code and thus encode the same protein as that encoded by the said nucleotide sequence.

In addition to the nucleotide sequence of any one of Seq ID No. 1 to 12 or a gene selected from the group consisting of Seq ID No 25 to 85, it will be appreciated by those skilled in the art that DNA sequence polymorphisms that lead to changes in the amino acid sequence may exist within a population. Such genetic polymorphisms may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Accordingly, in another embodiment, an isolated nucleic acid molecule of the invention is at least 300 (325, 350, 375, 400, 425, 450, 500, 550, 600, 650, 700, 800, 900, 1000, or 1290) nucleotides in length and hybridizes under stringent conditions to the nucleic acid molecule comprising the nucleotide sequence, preferably the coding sequence of any one of the genes listed in table 2 and/or 3 or Seq ID No. 25 to 85 and encodes an allelic variant or mutant of said gene.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y (1989), 6.3.1-6.3.6. A preferred, non-limiting example of stringent hybridization conditions are hybridization in 6xsodium chloride/sodium citrate (SSC) at about 45 degrees C., followed by one or more washes in 0.2× SSC, 0.1% SDS at 50-65 degrees C. Preferably, an isolated nucleic acid molecule of the invention that hybridizes under stringent conditions to the sequence selected from the group consisting of Seq ID No. 1 to 12 or a gene selected from the group consisting of Seq ID No 25 to 85, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally occurring allelic variants of any one of the genes listed in tables 2 and 3 or Seq ID No. 25 to 85, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologues of various species may be non-essential for activity and thus would be likely targets for alteration. Alternatively, amino acid residues that are conserved among the homologues of various species (e.g., murine and human) may be essential for activity and thus would not be likely targets for alteration.

Accordingly, another aspect of the invention pertains to nucleic acid molecules encoding a polypeptide selected from the group consisting of Seq ID No. 13 to 24 or a polypeptide selected from the group consisting of Seq ID No 86 to 146 that contain changes in amino acid residues that are not essential for activity. Such polypeptides differ in amino acid sequence from said polypeptide yet retain biological activity. In one embodiment, the isolated nucleic acid molecule includes a nucleotide sequence encoding a protein that includes an amino acid sequence that is at least about 85%, 95%, 96%, 97%, 98%, or 99% identical to the amino acid sequence of any one of Seq ID No. 13 to 24 or of Seq ID No. 86 to 146.

An isolated nucleic acid molecule encoding a variant protein can be created by introducing one or more nucleotide substitutions, additions or deletions into the nucleotide sequence of any one of the genes listed in tables 2 and 3 or Seq ID No.25 to 85 such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In one embodiment, a mutant polypeptide that is a variant of a polypeptide selected from the group consisting of Seq ID No. 13 to 24 or a polypeptide selected from the group consisting of Seq ID No 86 to 146 can be assayed for (1) the ability to form protein-protein interactions with proteins in a signaling pathway, (2) the ability to bind a ligand of said polypeptide; or (3) the ability to bind to an intracellular binding protein for said polypeptide. In another embodiment, the mutant polypeptide can be assayed for the ability to mediate changes in feeding behavior, body weight, or metabolism.

J The present invention encompasses antisense nucleic acid molecules, i.e., molecules which are complementary to a sense nucleic acid encoding a polypeptide selected from the group consisting of Seq ID No. 19 to 24 or a polypeptide selected from the group consisting of Seq ID No 99 to 146, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a noncoding region of the coding strand of a nucleotide sequence encoding a polypeptide selected from the group consisting of Seq ID No. 19 to 24 or a polypeptide selected from the group consisting of Seq ID No 99 to 146. The noncoding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiotiracil, 3-(3-amino-3-N-2-carboxypropyl) tiracil, (acp3) w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding uracil to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the anti-sense molecules, vector constructs in which the antisense nucleic acid molecule is placed under the control of a strong pol II or pol III promoter are preferred.

An antisense nucleic acid molecule of the invention can be an a-anomeric nucleic acid molecule. An a-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The anti-sense nucleic acid molecule can also comprise a 2′-o-methylribonucleotide (Inolle C et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of the protein encoded by the mRNA. A ribozyme having specificity for a nucleic acid molecule encoding a polypeptide selected from the group consisting of Seq ID No. 13 to 24 a polypeptide selected from the group consisting of Seq ID No 86 to 146 can be designed based upon the nucleotide sequence of a cDNA disclosed herein. For example, a derivative of a Tetrahymena L-19 JVS RNA can be constructed in which the nucleotide sequence of the active site is complementary to the nudeotide sequence to be cleaved. Cech et al. U.S. Pat. No.4,987,071; and Cech et al. U.S. Pat. No. 5,116,742. Alternatively, an mRNA encoding any one of the genes listed in tables 1 and 2 can be used to select a catalytic RNA having a specific ribonuclease activity from a pool of RNA molecules. See, e.g., Bartel and Szostak (1993) Science 261.1411-1418.

The invention also encompasses nucleic acid molecules which form triple helical structures. For example, expression of a polypeptide selected from the group consisting of Seq ID No. 19 to 24 or a polypeptide selected from the group consisting of Seq ID No 99 to 146, can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y Acad. Sci. 660:27-36; and Maher (1992) Bioassays 14(12):807-15.

In certain embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675. PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., 51 nucleases (Hyrup (1996), supra; or as probes or primers for DNA sequence and hybridization (Hyrup (1996), supra; Perry-O'Keefe et al. (1996) Proc. Natl. Acad. Sci. USA 93:1467˜675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Petersen et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89110134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

The present invention provides a use of a gene or a polypeptide encoded by a gene listed in tables 2 and/or 3 or Seq ID No. 25 to 85 as a target for screening of compounds that reduce and/or prevent obesity.

The present invention also provides a use of a nucleic acid encoding a polypeptide selected from the group consisting of Seq ID No. 13-24 a polypeptide selected from the group consisting of Seq ID No 86 to 146, or of mutants or fragments thereof as a target for screening of compounds that reduce and/or prevent obesity.

III. Isolated Proteins and Antibodies

One aspect of the invention pertains to isolated proteins, and biologically active portions thereof, as well as polypeptide fragments suitable for use as immunogen to raise antibodies directed against a polypeptide selected from the group consisting of Seq ID No. 13 to 24 or a polypeptide selected from the group consisting of Seq ID No 86 to 146. In one embodiment, native polypeptide can be isolated from cells or tissue sources by an appropriate purification scheme using standard protein purification techniques. In another embodiment, polypeptides of the invention are produced by recombinant DNA techniques. Alternative to recombinant expression, polypeptides can be synthesized chemically using standard peptide synthesis techniques. This section describes polypeptides of any one of Seq ID No. 13 to 24 or SeqID No. 86 to 146, antibodies directed against said polypeptides, and methods for making and using such polypeptides and antibodies.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or unrelated chemicals.

Biologically active portions of a polypeptide selected from the group consisting of Seq ID No. 13 to 24 or a polypeptide selected from the group consisting of Seq ID No 86 to 146 include polypeptides comprising amino acid sequences sufficiently identical to or derived from the amino acid sequence of the protein which include fewer amino acids than the full length protein, and exhibit at least one activity of the corresponding full-length protein. Typically, biologically active portions comprise a domain or motif with at least one activity of the corresponding portion. A biologically active portion of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of said polypeptide.

Among the useful polypeptides are those having the amino acid sequence of a polypeptide selected from the group consisting of Seq ID No. 13 to 24 or a polypeptide selected from the group consisting of Seq ID No 86 to 146. Other useful proteins are substantially identical (e.g., at least about 96%, 97%, 98%, 99%, or 99.5%) to any of said polypeptides and retain the functional activity of the protein of the corresponding naturally-occurring protein yet differ in amino acid sequence due to natural allelic variation or mutagenesis. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)xlOo). Preferably, the two sequences are the same length.

The invention also provides chimeric or fusion proteins. As used herein, a “chimeric protein” or “fusion protein” comprises all or part (e.g., biologically active fragment) of a polypeptide selected from the group consisting of one Seq ID No. 13 to 24 or a polypeptide selected from the group consisting of Seq ID No 86 to 146 operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of said polypeptide.

One useful fusion protein is a GST fusion protein in which all or a portion of a polypeptide selected from the group consisting of Seq ID No.13 to 24 or a polypeptide selected from the group consisting of Seq ID No 86 to 146 is fused to the C-terminus of GST sequences. Such fusion proteins can facilitate the purification of a recombinant polypeptide. Other useful fusion proteins include fusions to FLAGTM, a portion lacZ, GST, calmodulin-binding peptide, His⁶, or HA. Vectors for preparing such fusions proteins are available from Clontech, Inc. (Palo Alto, Calif.) and Stratagene, Inc. (La Jolla, Calif.).

In another embodiment, the fusion protein contains a heterologous signal sequence at its N-terminus. For example, the native signal sequence of any one of the polypeptides of Seq ID No. 13 to 24 or Seq ID No.86 o 146 can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook Ct al., supra) and the protein A secretory signal (Pharmacia Biotech; Piscataway, N.J.).

In yet another embodiment, the fusion protein is an immunoglobulin fusion protein in which all or part of the sequence of a polypeptide of Seq ID No. 13 to 24 or Seq ID No.86 to 146 is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of Seq ID No. 13 to 24 or Seq ID No. 86 to 146. Inhibition of ligand/receptor interaction may be useful therapeutically for modulating feeding behavior, body weight, and/or metabolic rate. Moreover, the immunoglobulin fusion proteins of the invention can be used as immunogen to produce antibodies directed against a polypeptide hereinbefore described in a subject, to purify ligands and in screening assays to identify molecules which inhibit the interaction of receptors with ligands.

Chimeric and fusion protein of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GSJ polypeptide). A nucleic acid encoding any one of the polypeptides of Seq ID No. 13 to 24 or Seq ID No. 86 to 146 can be cloned into such an expression vector such that the fusion moiety is linked in-frame to the polypeptide of the invention.

The present invention also pertains to variants of any one of the polypeptides of Seq ID No. 13 to 24 or Seq ID No. 86 to 146. Such variants have an altered amino acid sequence which can function as either agonists (mimetics) or as antagonist. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polyepeptides of the invention from a degenerate oligonucleotide sequence. Methods for synthesizing degenerate oligonucleotides are known in the art (see, e.g., Narang (1983) Tetrahedron 39:3; Itakura et al. (1984)Arniu. Rev. Biochem. 53:323; Itakura et al. (1984) Science 198:1056; Ike et al. (1983) Nucleic Acid Res. 11:477).

In addition, libraries of fragments of a polypeptide selected from the group consisting of Seq ID No. 13 to 24or a polypeptide selected from the group consisting of Seq ID No 86 to 146 can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected.

Recursive ensemble mutagenesis (REM), a technique which enhances the frequency of functional mutants in the libraries, can be used in combination with the screening assays to identify variants of a protein of the invention (Arkin and YollrvaIl (1992) Proc. Natl Acad USA 89:7811-7815; Delgrave et al. (1993) Protein Engineering 6(3):327-331).

IV. Recombinant Expression Vectors and Host Cells

Another aspect of the invention pertains to vectors (e.g., expression vectors) containing a nucleic acid encoding a polypeptide selected from the group consisting of Seq ID No. 13 to 24 or a polypeptide selected from the group consisting of Seq ID No 86 to 146 (or a portion thereof). As used herein, the “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions. This section describes vectors and host cells harboring nucleic acids selected from the group consisting of Seq ID No. 1 to 12 a gene selected from the group consisting of Seq ID No 25 to 85 and variants thereof and methods for their production and use.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the present invention can be designed for expression of a gene listed in tables 2 or 3 or Seq ID No. 25 to 85 in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include PGEX (Pharmacia Biotech Inc; Smith and Johnson (1988) Gene 67:31-40), pMAL (New England Biolabs, Beverly, Mass.) and pRIT5 (Pharmacia Piscataway, N.J.) which fuse glutathione 5-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET lid (Studier et al., Gene Expression Technology: Methods in Enzymology 185, Academic Press San Diego, Calif. (1990) 60˜9). Target gene expression from the pTrc vector relies on host RNA polymerase transcription from a hybrid trp-lac fusion promoter. Target gene expression from the pET lid vector relies on transcription from a T7 gn10-lac fusion promoter mediated by a coexpressed viral RNA polymerase (T7 gnl). The viral polymerase is supplied by host strains BL21(DE3) or HM5174 (DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSecl (Baldari et al. (1987) mEMBO J. 6:229-234), pMFa (Kurjan and Herskowitz, (1982) Cell 30:933˜943), pJRY88 (Schultz et al. (1987) Gene 54:113-123), pYES2 (Invitrogen Corporation, SanDiego, Calif.), and pPicZ (In Vitrogen Corp, San Diego, 15 Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol.20 3:2156-2165) and the pVL series (Lucklow and Summers (1989) virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elemen[s are ilsed to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No.4,873, 316 and European Application Publication No.264,166). Developmentally-regulated promoters are also encompassed, for example the murine hox promoters (Kessel and Gruss (1990) Science 249:374-379) and the CL-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide selected from the group consisting of Seq ID No. 13 to 24 or a polypeptide selected from the group consisting of Seq ID No 86 to 146. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activitvity of which can be determined by the cell type into which the vector is introduced. For a discussion of the regulation of gene expression using anti-sense genes see Weintraub et al. (Reviews—Trends in Genetics, Vol.1(1) 1986).

Another aspect of the invention pertains to host cells into which a recombinant expression vector of the invention has been introduced. It is understood that this termsrefer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell (e.g., E. coli, insect cells, yeast or mammalian cells). Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Useful selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide selected from the group consisting of Seq ID No. 13 to 24 or a polypeptide selected from the group consisting of Seq ID No 86 to 146. Accordingly, the invention further provides methods for producing a polypeptide selected from the group consisting of Seq ID No. 13 to 24 or a polypeptide selected from the group consisting of Seq ID No 86 to 146 using the host cells of the invention. In one embodiment, the method comprises culturing the host cell of invention (into which a recombinant expression vector encoding a polypeptide selected from the group consisting of Seq ID No. 13 to 24 or a polypeptide selected from the group consisting of Seq ID No 86 to 146 has been introduced) in a suitable medium such that the polypeptide is produced. In another embodiment, the method further comprises isolating the polypeptide from the medium or the host cell.

V. Methods of Treatment

The present invention provides for both prophylactic and therapeutic methods for modulating body weight, e.g., by altering feeding behavior or metabolic rate.

In one aspect, the present invention provides a method for modulating body weight by administering an agent which modulates an activity of any one of the polypeptides of Seq ID No. 13 to 24 or Seq ID No. 86 to 146. Such methods are useful for modulating body weight both in patients having aberrant expression or activity of said polypeptide or other patients which would benefit from administration of an agent which modulates activity of said polypeptide. Depending on the needs of the patient a polypeptide agonist or antagonist can be used for treating the subject.

Agonists of the activity of any one of the polypeptides of Seq ID No. 13-18 or Seq ID No. 86 to 146, or compounds which increase expression of said polypeptide are useful for treatment of high body weight, e.g., obesity, because they can be used to reduce body weight. Similarly, compounds which increase the activity or expression of a protein in the [symbol] ignaling pathway of said polypeptide are useful for treatment of high body weight. Conversely, antagonists of the activity of said polypeptide or compounds which reduce the expression or activity of said polypeptide are useful for treatment of low body weight, e.g., cachexia, because they can be used to increase body weight. Compounds which reduce the activity or expression of a protein in the [symbol] ignaling pathway of a polypeptide of any one of Seq ID NO. 13 to 18 or Seq ID No. 86 to 146 are useful for treatment of low body weight.

Antagonists of the activity of any one of the polypeptides of Seq ID No. 19-24 or Seq ID No. 99 to 146, or compounds which decrease expression of said polypeptide are useful for treatment of high body weight, e.g., obesity, because they can be used to reduce body weight. Similarly, compounds which decrease the activity or expression of a protein in the [symbol] ignaling pathway of said polypeptide are useful for treatment of high body weight. Conversely, agonists of the activity of said polypeptide or compounds which increase the expression or activity of said polypeptide are useful for treatment of low body weight, e.g., cachexia, because they can be used to increase body weight. Compounds which icrease the activity or expression of a protein in the [symbol] ignaling pathway of a polypeptide of any one of Seq ID NO. 19 to 24 or Seq ID No. 99 to 146 are useful for treatment of low body weight.

The present invention also provides a use of an agonist, or a compound which increases the expression of a polypeptide selected from the group consisting of Seq ID No. 13 tol8 or a polypeptide selected from the group consisting of Seq ID No 86 to 98 for the preparation of a medicament for the treatment of obesity. Futhermore, the present invention provides a use of an antagonist, or a compound that decreases the expression of a polypeptide selected from the group consisting of Seq ID No. 19 to 24 or a polypeptide selected from the group consisting of Seq ID No 99 to 146 for the preparation of a medicament for the treatment of obesity.

VI. Pharmaceutical Compositions

The present invention further pertains to novel agents identified by the above-described screening assays and uses thereof for treatments as described herein. The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or anti-body and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes pharmaceutical compositions comprising a modulator of expression or activity of any one of the polypeptides of Seq ID No. 13 to 24 or Seq ID No. 86 to 146 (and/or a modulator of the activity or expression of a protein in the signalling pathway of said polypeptide) as a well as methods for preparing such compositions by combining one or more such modulators and a pharmaceutically acceptable carrier. Also within the scope of the present invention are pharmaceutical compositions comprising a modulator identified using the screening assays of the invention packaged with instructions for use. For modulators that are antagonists of the activity of any one of the polypeptides of Seq ID No. 13 to 24 or Seq ID No. 86 to 146 or which reduce the expression of said polypeptide, the instructions would specify use of the pharmaceutical composition for treatment of low body weight (e.g., increase of body weight). For modulators that are agonists of the activity of said polypeptide or increase the expression of said polypeptide, the instructions would specify use of the pharmaceutical composition for treatment of high body weight (i.e., reduction of body weight).

The present invention provides a pharmaceutical formulation for the modulation of body weight, comprising a compound that modulates the activity of a polypeptide selected from the group consisting of Seq ID No. 86 to 146, mixed with a pharmaceutically acceptable carrier.

A pharmaceutical composition of the invention is formulated to be compatible with its intended route of administration. Examples of routes of administration include parenteral, e.g., intravenous, intradermal, subcutaneous, oral (e. g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions are provided. For intravenous administration, suitable carriers include physiological saline, bacteriostatic water, Cremophor ELTM (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No.4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to t)e achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

The present invention provides a pharmaceutical formulation for the modulation of body weight, comprising a compound that modulates the activity of a polypeptide selected from the group consisting of Seq ID No. 13 to 24 or a polypeptide selected from the group consisting of Seq ID No 86 to 146, mixed with a pharmaceutically acceptable carrier.

The present invention also refers to a package comprising the pharmaceutical formulation hereinbefore described and instructions for administering the pharmaceutical formulation for the purpose of modulating body weight.

The present invention pertains to a method for preparing a pharmaceutical composition useful for modulating body weight, the method comprising: a) contacting a test compound with a polypeptide selected from the group consisting of Seq ID No. 13 to 24 or a polypeptide selected from the group consisting of Seq ID No 86 to 146; b) determining whether the test compound binds to the polypeptide selected from the group consisting of Seq ID No. 13 to 24 or a polypeptide selected from the group consisting of Seq ID No 86 to 146; and c) combining the test compound that binds to the polypeptide selected from the group consisting of Seq ID No. 13 to 24 or a polypeptide selected from the group consisting of Seq ID No 86 to 146 with a pharmaceutically acceptable carrier to create a pharmaceutical composition useful for modulating body weight.

The present invention provides a method for preparing a pharmaceutical composition useful for modulating body weight, the method comprising: a) contacting a ligand of a polypeptide selected from the group consisting of Seq ID No.13 to 24 or a polypeptide selected from the group consisting of Seq ID No 86 to 146 with a polypeptide selected from the group consisting of Seq ID No.13 to 24 or a polypeptide selected from the group consisting of Seq ID No 86 to 146 in the presence and absence of a test compound; b) determining whether the test compound alters the binding of the ligand of the polypeptide selected from the group consisting of Seq ID No.13 to 24 or a polypeptide selected from the group consisting of Seq ID No 86 to 146 to the polypeptide selected from the group consisting of Seq ID No.13 to 24 or a polypeptide selected from the group consisting of Seq ID No 86 to 146; and c) combining the test compound that alters the binding of said ligand to said polypeptide with a pharmaceutically acceptable carrier to create a pharmaceutical composition useful for modulating body weight.

The present invention also provides a use of a gene selected from the group consisting of Seq ID No. 1 to 12 or a gene selected from the group consisting of Seq ID No 25 to 85, or of a polypeptide selected from the group consisting of Seq ID No 13 to 24 as a target for screening of compounds that reduce and/or prevent obesity.

The present invention further provides a use of a gene or a polypeptide encoded by a selected from the group consisting of Seq ID No.25 to 146 as a target for screening of compounds that reduce and/or prevent obesity.

Further to this, the present invention also pertains to a use of a nucleic acid encoding a polypeptide selected from the group consisting of Seq ID No.13-24, or of mutants or fragments thereof, as a target for screening of compounds that reduce and/or prevent obesity.

Furthermore, the present invention provides a use of a nucleic acid encoding a polypeptide selected from the group consisting of Seq ID No. 86 to 146, or of mutants or fragments thereof as a target for screening of compounds that reduce and/or prevent obesity.

The present invention also provides a kit for screening for compounds that reduce and/or prevent obesity comprising a polypeptide selected from the group consisting of Seq ID No. 13 to 24.

The present invention additionally provides a kit for screening for compounds that reduce and/or prevent obesity comprising a polypeptide selected from the group consisting of Seq ID No. 86 to 146.

### EXAMPLES

### Example 1

### RNA Preparation

Total RNA from 500 mg subcutaneous fat tissue was isolated using the TriZol reagent (Life Technologies) and the Fast RNA green (BIO101) kit according to the manufacturer's protocols. Total DNA was purified from contaminating DNA the RNeasy kit (Qiagen).

### Example 2

### Gene Expression Measurement by DNA Chips

Synthesis of first and second strand cDNA were performed using the SuperScript Choice Gene Chip Kit (Life Technologies) and reagents from Gibco. The double stranded cDNA, containing an incorporated T7 RNA polymerase binding site, was purified by extraction with a mix of phenol:chloroform:isoamylalcohol (Life Technologies). The organic and aqueous phases were separated by Phase Lock Gel (Eppendorf) and double-stranded cDNA was recovered by precipitation according to the manufacturer's protocol and then resuspended in water.

J The double-stranded cDNA was converted to biotin-labeled cRNA by in vitro transcription (IVT) using a T7 kit (Ambion) and biotin-containing ribonucleotides (Enzo—LOXO GmbH). The IVT-material was purified from unincorporated ribonucleotides using RNeasy spin columns (Qiagen). Following cleanup, the single-stranded biotin-labeled cRNA were chemically hydrolyzed to smaller fragments in 500 mM calcium acetate, 150 mM magnesium acetate, pH 8.1 for 35 min at 95° C. The reaction was terminated by chilling samples on ice.

Probes were hybridized to the U95 A GeneChip Microarray (Affymetrix) which contains features representing ˜12,000 genes. All washing, hybridization, detection, and signal amplification steps were performed using a GeneChip Fluidics Station (Affymetrix). Fluorescence intensity data was collected from the hybridized GeneArrays using a GeneArray scanner (Affymetrix). The raw files containing the fluorescence intensity information were transformed into data files using the Affymetrix Microarray Suite (MAS) software. Differentially expressed genes were identified using the Roche Affymetrix Chip Experiment Analysis (RACE-A) software. Differences between control patients (n=10) vs. obese case patients (n=10) were evaluated by several statistical filters as change factor vs. control.

TABLE 2
Genes down-regulated in fat tissue in obesity

| | | | | | | Unigene |
|---|---|---|---|---|---|---|
| Seq ID No. | | | | | | No. |
| DNA | | | | Lean | Obese | Accession |
| (protein) | Description | CHCF | P value | mean | mean | No. |
| 1 (13) | Alpha-2-glycoprotein, | −10.57 | 0.01292 | 122.29 | 10.35 | His.512643 |
| | zinc | | | | | NM001185 |
| 2 (14) | Insulin receptor substrate 1 | −1.25 | 0.00211 | 21.64 | 8.51 | Hs.390242 |
| | | | | | | NM005544 |
| 3 (15) | Apolipoprotein a-i | −418.62 | 0.05987 | 1678.49 | 621.08 | Hs.93194 |
| | | | | | | M27875 |
| 4 (16) | Aryl hydrocarbon | −1.14 | 0.00032 | 30.9 | 13.49 | Hs.131494 |
| | receptor nuclear | | | | | NM001668 |
| | translocator | | | | | |
| 5 (17) | Creatine kinase, brain | −0.82 | 0.01454 | 395.52 | 223.65 | Hs.173724 |
| | | | | | | NM001823 |
| 6 (18) | Sterol-c5-desaturase | −1.13 | 0.00011 | 48.56 | 27.03 | Hs.287749 |
| | | | | | | AF187981 |

TABLE 3
Genes up-regulated in fat tissue in obesity

| Seq ID | | | | | | Unigene |
|---|---|---|---|---|---|---|
| No. | | | | | | No. |
| DNA | | | | Lean | Obese | Accession |
| (protein) | Description | CHCF | P value | mean | mean | No. |
| 7 (19) | osteomodulin | 1.1 | 0.00008 | 2.96 | 7.77 | Hs.94070 |
| | | | | | | NM005014 |
| 8 (20) | Very low density | 1.65 | 0.0032 | 67.14 | 96.66 | Hs.370422 |
| | lipoprotein receptor | | | | | D16494 |
| 9 (21) | Hydroxysteroid (11-beta) | 1.87 | 0.00108 | 25.78 | 58.23 | Hs.275215 |
| | dehydrogenase 1 | | | | | NM005525 |
| 10 (22) | reelin | 1.72 | 0.00003 | 4.79 | 11.83 | Hs.431010 |
| | | | | | | U79716 |
| 11 (23) | Atp-binding cassette, sub- | 1.1 | 0.00193 | 17.71 | 38.51 | Hs.55879 |
| | family c (cftr/mrp), | | | | | AL133613 |
| | member 10 | | | | | |
| 12 (24) | Homo sapiens dna for | 2.63 | 0.00008 | 53.63 | 145.18 | D63861 |
| | cyclophilin 40 | | | | | |

TABLE 4
Genes down-regulated in fat tissue in obesity

| Seq | | | | Locus | |
|---|---|---|---|---|---|
| ID No. | | | | ID | |
| DNA | | | P | (Gene | Accession |
| (protein) | Description | CHCF | value | ID) | No. |
| 2 | Retinoid | — | 0.000 | 625 | NM_0069 |
| 5 (86) | x receptor | 0.63 | 41 | 8 | 17 |
| | gamma | | | | |
| 26 (87) | Vascular | −1.52 | 0.01468 | 7422 | NM_003376 |
| | endothelial | | | | |
| | growth factor | | | | |
| 27 (88) | Cannabinoid | −0.34 | 0.00788 | 1268 | NM_016083 |
| | receptor 1 | | | | Variant 1 |
| | (brain) | | | | |
| 28 (89) | | | | | NM_033181 |
| | | | | | Variant 2 |
| 29 (90) | Cathepsin O | −0.52 | 0.00260 | 1519 | NM_001334 |
| 30 (91) | Enoyl | −0.45 | 0.00169 | 1892 | NM_004092 |
| | coenzyme | | | | |
| | a hydratase, | | | | |
| | short | | | | |
| | chain 1, | | | | |
| | mitochondrial | | | | |
| 31 (92) | Vascular | −0.24 | 0.00169 | 7423 | NM_003377 |
| | endothelial | | | | |
| | growth factor B | | | | |
| 32 (93) | Pyruvate | −0.77 | 0.00520 | 5091 | NM_000920 |
| | carboxylase | | | | Variant A |
| 33 (94) | | | | | NM_022172 |
| | | | | | Variant 2 |
| 34 (95) | L-3-hydroxy- | −0.83 | 0.00201 | 3033 | NM_005327 |
| | acyl | | | | |
| | Coenzyme A | | | | |
| | dehydrogenase, | | | | |
| | short chain | | | | |
| 35 (96) | pyruvate | −0.47 | 0.00186 | 5164 | NM_002611 |
| | dehydrogenase | | | | |
| | kinase, | | | | |
| | isoenzyme 2 | | | | |
| 36 (97) | Citrate | −0.66 | 0.02528 | 1431 | NM_004077 |
| | synthase | | | | Variant 1 |
| 37 (98) | | | | | NM_198324 |
| | | | | | Variant 2 |

TABLE 5
Genes up-regulated in fat tissue in obesity

| Seq ID | | | | | |
|---|---|---|---|---|---|
| No. | | | | | |
| DNA | | | | Locus ID | |
| (protein) | Description | CHCF | P value | (Gene ID) | Accession No. |
| 38 (99) | fibroblast growth factor | 1.12 | 0.00015 | 2263 | NM_000141 |
| | receptor 2 | | | | Variant 1 |
| 39 (100) | | | | | NM_022969 |
| | | | | | Variant 2 |
| 40 (101) | | | | | NM_022970 |
| | | | | | Variant 3 |
| 41 (102) | | | | | NM_022971 |
| | | | | | Variant 4 |
| 42 (103) | | | | | NM_022972 |
| | | | | | Variant 5 |
| 43 (104) | | | | | NM_022973 |
| | | | | | Variant 6 |
| 44 (105) | | | | | NM_022974 |
| | | | | | Variant 7 |
| 45 (106) | | | | | NM_022975 |
| | | | | | Variant 8 |
| 46 (107) | | | | | NM_022976 |
| | | | | | Variant 9 |
| 47 (108) | | | | | NM_023028 |
| | | | | | Variant 10 |
| 48 (109) | | | | | NM_023029 |
| | | | | | Variant 11 |
| 49 (110) | | | | | NM_023030 |
| | | | | | Variant 12 |
| 50 (111) | | | | | NM_023031 |
| | | | | | Variant 13 |
| 51 (112) | tumor necrosis factor receptor | 0.32 | 0.00015 | 7133 | NM_001066 |
| | superfamily, member 1B | | | | |
| 52 (113) | tumor necrosis factor (ligand) | 0.75 | 0.00548 | 8743 | NM_003810 |
| | superfamily, member 10 | | | | |
| 53 (114) | angiopoietin 1 | 1.52 | 0.00010 | 284 | NM_001146 |
| | | | | | Variant 1 |
| 54 (115) | | | | | NM_139290 |
| | | | | | Variant 2 |
| 55 (116) | retinoid X receptor, alpha | 0.42 | 0.00885 | 6256 | NM_002957 |
| 56 (117) | degenerative spermatocyte | 1.19 | 0.00011 | 8560 | NM_003676 |
| | homolog, lipid desaturase | | | | Variant 1 |
| 57 (118) | | | | | NM_144780 |
| | | | | | Variant2 |
| 58 (119) | solute carrier family 7 member 7 | 1.15 | 0.00215 | 9056 | NM_003982 |
| 59 (120) | procollagen-lysine, 2- | | | | NM_000935 |
| | oxoglutarate 5-dioxygenase | | | | Variant 2 |
| 60 (121) | 2 | | | | NM_182943 |
| | | | | | Variant 1 |
| 61 (122) | UDP-glucose dehydrogenase | 0.64 | 0.00045 | 7358 | NM_003359 |
| 62 (123) | phosphatidylinositol-4- | 0.43 | 0.00892 | 8396 | NM_003559 |
| | phosphate 5-kinase, type II | | | | Variant 1 |
| 63 (124) | beta | | | | NM_138687 |
| | | | | | Variant 2 |
| 64 (125) | MAP kinase interacting | 0.64 | 0.00013 | 2872 | NM_017572 |
| | serine/threonine kinase 2 | | | | Sequence 1 |
| 65 (126) | | | | | NM_199054 |
| | | | | | Sequence 2 |
| 66 (127) | carboxypeptidase E | 1.52 | 0.00013 | 1363 | NM_001873 |
| 67 (128) | carboxypeptidase A3 (mast | 0.8 | 0.00802 | 1359 | NM_001870 |
| | cell) | | | | |
| 68 (129) | solute carrier family 38, | 1.45 | 0.00271 | 145389 | NM_153811 |
| | member 6 | | | | |
| 69 (130) | very low density lipoprotein | 1.65 | 0.00320 | 7436 | NM_003383 |
| | receptor | | | | |
| 70 (131) | cathepsin G | 1.61 | 0.00057 | 1511 | NM_001911 |
| 71 (132) | Galactokinase 2 | 1.02 | 0.00067 | 2585 | NM_001001556 |
| | | | | | Variant 2 |
| 72 (133) | | | | | NM_002044 |
| | | | | | Variant 1 |
| 73 (134) | prostaglandin D2 synthase | 1 | 0.00046 | 5730 | NM_000954 |
| 74 (135) | mitogen-activated protein | 0.86 | 0.00014 | 5601 | NM_002752 |
| | kinase 9 | | | | Variant 1 |
| 75 (136) | | | | | NM_139068 |
| | | | | | Variant 2 |
| 76 (137) | | | | | NM_139069 |
| | | | | | Variant 3 |
| 77 (138) | | | | | NM_139070 |
| | | | | | Variant 4 |
| 78 (139) | Interleukin 15 | 0.95 | 0.00007 | 3600 | NM_000585 |
| | | | | | Variant 3 |
| 79 (140) | | | | | NM_172174 |
| | | | | | Variant 1 |
| 80 (141) | | | | | NM_172175 |
| | | | | | Variant 2 |
| 81 (142) | mucosa associated lymphoid | 1.24 | 0.00380 | 10892 | NM_006785 |
| | tissue lymphoma translocation | | | | Variant 1 |
| 82 (143) | gene 1 | | | | NM_173844 |
| | | | | | Variant2 |
| 83 (144) | lysyl oxidase | 0.7 | 0.04940 | 4015 | NM_002317 |
| 84 (145) | Integrin beta 5 | 1.38 | 0.00030 | 3693 | NM_002213 |
| 85 (146) | aldehyde dehydrogenase 3 | 0.91 | 0.00003 | 224 | NM_000382 |
| | family, member A2 | | | | |

1. A method of screening for compounds that reduce and/or prevent obesity comprising
a) contacting a cell expressing a gene selected from the group consisting of Seq. ID Nos. 1-6 with a compound;
b) measuring the expression of said gene, or the expression of a polypeptide encoded by said gene; and
c) determining whether the compound up-regulates the expression of said gene or of the polypeptide encoded by said gene.

2. The method of claim 1, wherein the gene is Seq. ID No. 1.

3. The method of claim 1, wherein the gene is Seq. ID No. 2.

4. The method of claim 1, wherein the gene is Seq. ID No. 3.

5. The method of claim 1, wherein the gene is Seq. ID No. 4.

6. The method of claim 1, wherein the gene is Seq. ID No. 5.

7. The method of claim 1, wherein the gene is Seq. ID No. 6.

8. A method of screening for compounds that reduce and/or prevent obesity comprising
a) contacting a cell expressing a gene selected from the group consisting of Seq ID No. 25-37 with a compound;
b) measuring the expression of said gene, or the expression of a polypeptide encoded by said gene; and
c) determining whether the compound up-regulates the expression of said gene or of the polypeptide encoded by said gene.

9. A method of screening for compounds that reduce and/or prevent obesity comprising
a) contacting a cell expressing a gene selected from the group consisting of Seq. ID Nos. 7-12 with a compound;
b) measuring the expression of said gene, or the expression of a polypeptide encoded by said gene; and
c) determining whether the compound down-regulates the expression of said gene or of a polypeptide encoded by said gene.

10. The method of claim 8, wherein the gene is Seq. ID No. 7.

11. The method of claim 8, wherein the gene is Seq. ID No. 8.

12. The method of claim 8, wherein the gene is Seq. ID No. 9.

13. The method of claim 8, wherein the gene is Seq. ID No. 10.

14. The method of claim 8, wherein the gene is Seq. ID No. 11.

15. The method of claim 8, wherein the gene is Seq. ID No. 12.

16. A method of screening for compounds that reduce and/or prevent obesity comprising
a) contacting a cell expressing a gene selected from the group consisting of Seq ID No. 38 to 85 with a compound;
b) measuring the expression of said gene, or the expression of a polypeptide encoded by said gene; and
c) determining whether the compound down-regulates the expression of said gene or of a polypeptide encoded by said gene.

17. A method of screening for compounds that reduce and/or prevent obesity comprising: a) contacting a polypeptide selected from the group consisting of Seq ID No. 13 to 18 with a compound; and
b) determining the activity of said polypeptide;
wherein a compound which reduces and/or prevents obesity by agonizing said polypeptide is a compound which causes an increase in activity of said polypeptide.

18. A method of screening for compounds that reduce and/or prevent obesity comprising: a) contacting a polypeptide selected from the group consisting of Seq ID No. 86 to 98 with a compound; and
b) determining the activity of said polypeptide;
wherein a compound which reduces and/or prevents obesity by agonizing said polypeptide is a compound which causes an increase in activity of said polypeptide.

19. A method of screening for compounds that reduce and/or prevent obesity comprising: a) contacting a polypeptide selected from the group consisting of Seq ID No. 19 to 24 with a compound; and
b) determining the activity of said polypeptide;
wherein a compound which reduces and/or prevents obesity by antagonizing said polypeptide is a compound which causes a decrease in activity of said polypeptide.

20. A method of screening for compounds that reduce and/or prevent obesity comprising: a) contacting a polypeptide selected from the group consisting of Seq ID No. 99 to 146 with a compound; and
b) determining the activity of said polypeptide;
wherein a compound which reduces and/or prevents obesity by antagonizing said polypeptide is a compound which causes a decrease in activity of said polypeptide.

21. A method for screening of compounds that reduce and/or prevent obesity comprising: a) contacting a cell expressing a nucleic acid encoding a polypeptide selected from the group consisting of Seq ID No. 13 to 18 with a compound; and
b) determining the activity of said polypeptide;
wherein a compound which reduces and/or prevents obesity by agonizing said polypeptide is a compound which causes an increase in activity of said polypeptide.

22. A method for screening of compounds that reduce and/or prevent obesity comprising: a) contacting a cell expressing a nucleic acid encoding a polypeptide selected from the group consisting of Seq ID No.86 to 98 with a compound; and
b) determining the activity of said polypeptide;
wherein a compound which reduces and/or prevents obesity by agonizing said polypeptide is a compound which causes an increase in activity of said polypeptide.

23. A method for screening of compounds that reduce and/or prevent obesity comprising: a) contacting a cell expressing a nucleic acid encoding a polypeptide selected from the group consisting of Seq ID No. 19 to 24 with a compound; and
b) determining the activity of said polypeptide;
wherein a compound which reduces and/or prevents obesity by antagonizing said polypeptide is a compound which causes a decrease in activity of said polypeptide.

24. A method for screening of compounds that reduce and/or prevent obesity comprising: a) contacting a cell expressing a nucleic acid encoding a polypeptide selected from the group consisting of Seq ID No. 99 to 146 with a compound; and
b) determining the activity of said polypeptide;
wherein a compound which reduces and/or prevents obesity by antagonizing said polypeptide is a compound which causes a decrease in activity of said polypeptide.

25. A method of screening for compounds that bind to a polypeptide selected from the group consisting of the polypeptides of Seq ID No. 13 to 24, comprising the steps of
a) contacting a compound with said polypeptide; and
b) determining the ability of said compound to bind to said polypeptide.

26. A method of screening for compounds that bind to a polypeptide selected from the group consisting of the polypeptides of Seq ID No.86 to 146, comprising the steps of
a) contacting a compound with said polypeptide; and
b) determining the ability of said compound to bind to said polypeptide.

34. A pharmaceutical composition for the modulation of body weight, comprising a compound that modulates the expression or activity of a polypeptide selected from the group consisting of Seq ID No. 13 to 24, mixed with a pharmaceutically acceptable carrier.

35. A pharmaceutical composition for the modulation of body weight, comprising a compound that modulates the expression or activity of a polypeptide selected from the group consisting of Seq ID No. 86 to 146, mixed with a pharmaceutically acceptable carrier.
